(19) United States
(12) Patent Application Publication
(10) Pub. No.: US 2025/0289907 A1
(43) Pub. Date: Sep. 18, 2025

(54) PHOSPHATIDYLSERINE TARGETING MOLECULE COMBINED WITH MEMORY T CELL ANTIGEN FOR THERAPEUTIC, DIAGNOSTIC AND ADJUVANT USE

(71) Applicant: Rutgers, The State University of New Jersey, New Brunswick, NJ (US)

(72) Inventor: Alok K. Choudhary, Plainsboro, NJ (US)

(73) Assignee: Rutgers, The State University of New Jersey, New Brunswick, NJ (US)

(21) Appl. No.: 19/079,215

(22) Filed: Mar. 13, 2025

Related U.S. Application Data
(60) Provisional application No. 63/564,749, filed on Mar. 13, 2024.

(51) Int. Cl. C07K 16/44; A61K 38/17; A61K 39/00; A61K 39/04; A61K 39/215; A61K 45/06; A61P 31/06; A61P 37/04; G01N 33/569; G01N 33/68

(52) U.S. Cl. CPC C07K 16/44; A61K 38/17; A61K 39/04; A61K 39/215; A61K 45/06; A61P 31/06; A61P 37/04; G01N 33/5695; G01N 33/6866; A61K 2039/545; G01N 2333/57

(57) ABSTRACT

Phosphatidylserine (PS)-targeting biomolecules and related methods for impacting the function of memory T cells are provided. In the present methods, PS-binding biomolecules are delivered to peripheral blood mononuclear cells (PBMCs) or whole blood of a subject in the presence of antigens specific to one or more diseases, wherein the PBMCs or whole blood comprise memory T cells that are exhausted or non-responsive to the antigens specific to the one or more diseases. Delivery of the PS-binding biomolecules to the PBMCs or whole blood restores effector function in the antigen non-responsive memory T cells. The administration of PS-binding biomolecules to PBMCs or blood with T cell antigens specific to pathogens can also be used to develop ultrasensitive diagnostics for latent infections or memory T cells response induced by natural infection or vaccination.

### CROSS-REFERENCE TO RELATED APPLICATION

The present application claims priority to U.S. Provisional Patent Application No.

63/564,749 filed on Mar. 13, 2024, which is incorporated herein by reference in its entirety.

### TECHNICAL FIELD

The present application relates to synthetic or biomolecules and more specifically phosphatidylserine-targeting synthetic or biomolecules and related methods for impacting the function of memory T cells.

### BACKGROUND

The externalization of phosphatidylserine (PS) on the cell surface is an important marker of apoptosis, where its exposure facilitates the interaction with receptors like MerTK and Axl on macrophages for silent clearance of apoptotic cells. This process is evolutionarily conserved, as observed in several species (1) (2) (3) (4) (5). However, PS externalization is not exclusive to apoptotic cells, as it also occurs in live cells like platelets (6), neutrophils (7), B cells, (8), and T-cell (9). Live cells exhibit PS externalization in response to certain signals, which does not lead to their immediate engulfment by macrophages (10). This is thought to result from different activation pathways, which, unlike apoptosis, trigger the same phospholipid scramblase that externalizes PS (11). This differentiation in PS externalization between apoptotic and live cells is key to understanding their specific function, immune regulation, and cell turnover. These include enhancing the coagulation cascade in platelets (12), modulating granular exocytosis in activated mast cells and neutrophils (13), facilitating erythrocyte adhesion to endothelial surfaces (14), and contributing to the formation of immunological synapses between T cells and antigen-presenting cells (APCs) (15). Additionally, PS exposure has been shown to increase resistance to antigen-induced activation in B cells (16). Notably, extracellular PS suppresses T cell activity through its interaction with G protein-coupled receptor 174 (GPR174) on T cells (17). Furthermore, PS-binding molecules have been shown to enhance antitumor T cell responses by targeting immunosuppressive exosomes in the human tumor microenvironment (18). Despite these findings, the role of activation-induced PS exposure in T cell function remains poorly understood, particularly in the context of PS-targeting antibodies induced during infections or in autoimmune diseases (19) (20-23) (24) (25) (26) (27, 28) (29, 30).

### SUMMARY

In a first aspect, a method of restoring effector function in antigen non-responsive or exhausted memory T cells is provided. In the method, a therapeutically effective amount of phosphatidylserine (PS)-binding biomolecules is delivered to one or more peripheral blood mononuclear cells (PBMCs) or whole blood of a subject in the presence of antigens specific to one or more diseases or neoantigens specific to cancer cells. The PBMCs or whole blood comprise memory T cells that are exhausted or non-responsive to the antigens specific to the disease or the neoantigens specific to cancer cells, and delivery of the PS-binding biomolecules to the PBMCs or the whole blood restores effector function in the antigen-nonresponsive or neoantigen-nonresponsive memory T cells.

In another aspect, the increase in effector function of the memory T cells is quantified.

In another aspect, the subject is exposed to Mycobacterium tuberculosis (M.tb), and the antigens are M.tb-specific antigens.

In another aspect, the memory T cells are CD4+ memory T cells or CD8+ memory T cells.

In another aspect, the subject has previously taken a vaccine for the disease.

In another aspect, the disease is caused by SARS-CoV2 or Mycobacterium tuberculosis.

In another aspect, the PS-binding biomolecules are antibodies and wherein the antibodies are monoclonal antibodies or polyclonal antibodies.

In another aspect, the PS-binding biomolecules are annexins or synthetic small molecules.

In a second aspect, a method of treating one or more latent infections in a subject is provided. In the method, a therapeutically effective amount of phosphatidylserine (PS)-binding biomolecules is administered to the subject in the presence of antigens specific to one or more diseases that cause the one or more latent infections. The administration of the PS-binding biomolecules restores effector function in memory T cells of the subject that are exhausted or non-responsive to the disease-specific antigens.

In another aspect, the latent infection is caused by Mycobacterium tuberculosis (M.tb).

In another aspect, the subject is co-infected by HIV and M.tb.

In another aspect, the memory T cells are CD4+ memory T cells or CD8+ memory T cells.

In another aspect, the PS-binding biomolecules are antibodies and wherein the antibodies are monoclonal antibodies or polyclonal antibodies.

In a further aspect, the therapeutically effective amount of the antibodies is approximately 100-200 mg/ml.

In another aspect, the PS-binding biomolecules are annexins or synthetic small molecules.

In a third aspect, a method of restoring effector function in antigen non-responsive or exhausted memory T cells of a subject is provided. In the method, a blood sample is first obtained. A therapeutically effective amount of phosphatidylserine (PS)-binding biomolecules is then delivered to the blood sample in the presence of antigens specific to one or more diseases or neoantigens specific to cancer cells. The blood sample comprising the phosphatidylserine (PS)-binding biomolecules is then transfused in the presence of the antigens or neoantigens to the subject, and the transfused blood sample restores effector function in memory T cells of the subject that are exhausted or non-responsive to the antigens specific to one or more diseases or the neoantigens specific to cancer cells.

In another aspect, the antigens are M.tb-specific antigens.

In another aspect, the antigens are neoantigens.

In another aspect, the PS-binding biomolecules are antibodies and wherein the antibodies are monoclonal antibodies or polyclonal antibodies.

In another aspect, wherein the PS-binding biomolecules are annexins or synthetic small molecules.

In a fourth aspect, a method of diagnosing a latent Mycobacterium tuberculosis (M.tb) infection in a subject is provided. In the method, a blood sample of a subject is obtained, wherein the blood sample comprises memory T cells. The blood sample of the subject is then assayed with PS-binding biomolecules in the presence of M.tb-specific antigens. A level of IFNγ in the blood sample is measured via the assay, and based on the level of IFNγ in the assay, it is determined whether the subject has a latent M.tb infection.

In another aspect, if the level of IFNγ in the blood sample is IFNγ≥0.35 IU/ml and ≥25% of Nil, the subject has a latent M.tb infection.

In another aspect, the assay is a QuantiFERON-TB Gold Plus assay, an Interferon Gamma Release Assay (IGRA), or an ELISPOT assay.

In another aspect, the memory T cells are CD4+ memory T cells or CD8+ memory T cells.

In another aspect, the PS-binding biomolecules are antibodies. In a further aspect, the antibodies are monoclonal antibodies. In a further aspect, the antibodies are polyclonal antibodies.

In another aspect, the PS-binding biomolecules are annexins.

In another aspect, the PS-binding biomolecules are synthetic small molecules.

In a fifth aspect, a method of enhancing CD4/CD8 memory T cell response in a subject with a latent Mycobacterium tuberculosis (M.tb) infection is provided. In the method, a therapeutically effective amount of PS-binding molecules is administered to the subject with the M.tb infection in the presence of M.tb-specific antigens.

In another aspect, the memory T cells are CD4+ memory T cells or CD8+ memory T cells.

In another aspect, the PS-binding biomolecules are antibodies. In a further aspect, the antibodies are monoclonal antibodies. In a further aspect, the antibodies are polyclonal antibodies.

In another aspect, the PS-binding biomolecules are annexins.

In another aspect, the PS-binding biomolecules are synthetic small molecules.

In another aspect, the subject is co-infected with M.tb and HIV-1 and wherein the PS-binding molecules are administered in the presence of M.tb-specific and HIV-1 specific antigens.

### BRIEF DESCRIPTION OF THE DRAWINGS

FIG. 1. Fresh isolated PBMCs from COVID19 convalescent individual were activated with CD3/28, CoV2 Spike peptide T cell antigens (Ag) in the presence and absence of 11.31 mAb (100 ug/ml) O/N with brefeldin. Cells were stained for CD3+FITC cell surface marker and intracellular IFNγ-AF647 and TNFα-AF647. Significant increase in the IFNγ and TNFα level were detected by Ag-stimulated memory T treated with 11.31 mAb (SEQ ID NO. 1), while the just opposite effect was detected with the CD3/28 stimulated T cells.

FIGS. 2A-2C. FIG. 2A: PBMCs isolated from solid organ transplant recipients with clinically suppressed T cells with history of COVID-19 infection/vaccination were tested with (+) and without (−) PS mAb to detect the three different set of SARS-CoV2 peptide Ags for T cells S(1+2), S2N and SNMO [SARS-CoV2 spike protein(S) is composed of two subunits S1 and S2, (N) nucleocapsid; membrane protein (M) and (0) refers to ORF3-a and ORF-7a] FIG. 2B: CD3/28 and PHA activation with (+) and without (−) PS mAb. FIG. 2C: SARS-CoV2 Ags S(1+2) [mixture of T cell peptides representing S1 and S2] and S2N [mixture of T cell peptides representing S2 and N] were tested with three different concentration of PS mAb. NIL refers to no Ag added to the well.

FIGS. 3A-3E. Represents the IGRA results of five different individuals. Each graph represents eight bars as the level of IFNγ from two sets of four tubes labeled TB1, TB2, PHA, and NIL without (−) added PS mAb and with (+) PS mAb.

FIGS. 4A-4C. M.tb growth in four different human PBMCs with and without PS mAb. Two LTBI diagnosed (FIGS. 4B and 4C) and one healthy (FIG. 4A) PBMCs with unknown LTBI diagnosis were used. PS mAb was used at different concentration.

FIGS. 5A-5D. Four different Prot-G purified polyclonal plasma Abs and flow-through binding to PS (FIG. 5A), their competition with Biot-anti-PS-mAb binding to PS (FIG. 5B), their effect on CD3/28 activated T cells (FIG. 5C) and, common vaccine Ag activated memory T cells (FIG. 5D).

FIGS. 6A-6J. HIV-1 infected (left panel) and TB infected (right panel) plasma Ab binding to PS (FIGS. 6A-6B), CL (FIGS. 6C-6D) and β2GP (FIGS. 6E-6F) detected by ELISA. A positive correlation between plasma-PS and CL Abs (FIGS. 6G-6H) and PS and CL plasma Ab titer in PTB and LTB patients (FIGS. 6I-6J).

FIGS. 7A-7B. PD1 double-positive CD4 and CD8 T cell subpopulations and their frequencies are shown using bold, italics, and regular fonts. “Bold” indicates an increase, “italics” indicates a decrease, and “regular” indicates no difference in the percentage frequency of those cells when treated with the PS antibody compared to untreated cells. The table of FIG. 7A represents the unactivated, antigen-activated, and CD3/28 antibody-stimulated CD4 T cell populations, while table at FIG. 7B represents the unactivated, antigen-activated, and CD3/28 antibody-stimulated CD8 T cell populations. Due to the smaller number of cells in the naïve-like and central memory CD8 T cell populations among CD3/28 activated cells, the PD1 double-positive cells in these populations were not able to be analyzed.

FIG. 7C. Frequency of IFNγ positive CD4 and CD8 T cells subpopulation upon antigen and CD3 activation in the presence and absence of PS antibody. TN: Naïve T Cells, TSCM: Stem Cells like Memory T Cells, TCM: Central Memory T Cells, TEMRA: Terminal Differentiated T Cells Expressing CD45RA, TEM: Effector Memory T Cells.

### DETAILED DESCRIPTION

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

As used herein, the terms “antibody” and “antibodies” include polyclonal antibodies and/or monoclonal antibodies. Polyclonal antibodies are heterogeneous populations of antibody molecules that are specific for a particular antigen, while monoclonal antibodies are homogeneous populations of antibodies to a particular epitope contained within an antigen. As used herein, the term “anti-phosphatidylserine antibody” or “anti-PS antibody” refers to an antibody that binds to an inhibits phosphatidylserine on the surface of cells.

“CD3” (cluster of differentiation 3) generally refers to a protein complex and T cell co-receptor that is involved in activation of the cytotoxic T cell (CD8+ naive T cells) and T helper cells (CD4+ naive T cells).

“CD28” (Cluster of Differentiation 28) generally refers to the proteins expressed on T cells that provide co-stimulatory signals required for T cell activation and survival.

The term “Host-directed therapies” (HDT) generally refers to interventions that aim to improve the body's immune response against microbial pathogens or prevent tumorigenesis by targeting intracellular, innate, or adaptive immune reactions, or by preventing harmful immune responses.

The term “memory T cell” generally refers to antigen-specific T cells that remain in the body of a subject long after an infection by a pathogen has been cleared by the body. Memory T cells can be either CD4+ memory T cells or CD8+ memory T cells.

The term “peptide”, as used herein, refers to peptides and proteins longer than two amino acids in length that may also incorporate non-amino acid molecules.

The phrases “pharmaceutically acceptable” or “pharmacologically acceptable” refer to molecular entities and compositions that do not produce an adverse, toxic, allergic, inflammatory, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like that are pharmaceutically acceptable as the term is used herein and preferably inert. The use of such media and agents for pharmaceutical active substances is well known in the art. Supplementary active ingredients can also be incorporated into the compositions. Except insofar as any conventional media or agent is incompatible with the active ingredients, its use in therapeutic compositions is contemplated.

The term “phosphatidylserine” (PS) refers to a phospholipid that is a component of the cell membrane that impacts cell cycle signaling, specifically as it relates to apoptosis (a type of programmed cell death). Specifically, exposure of PS on the outer surface of the cell membrane can mark the cell for subsequent apoptosis.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the disclosure can be administered. In an exemplary embodiment of the present disclosure, to identify subject patients for treatment according to the methods of the disclosure, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of the present disclosure.

The terms “treat”, “treating” or “treatment” of a state, disorder or condition includes: (a) preventing or delaying the appearance of clinical symptoms of the state, disorder, or condition developing in a person who may be afflicted with or predisposed to the state, disorder or condition but does not yet experience or display clinical symptoms of the state, disorder or condition; or (b) inhibiting the state, disorder or condition, i.e., arresting, reducing or delaying the development of the disease or a relapse thereof (in case of maintenance treatment) or at least one clinical symptom, sign, or test, thereof; or (c) relieving the disease, i.e., causing regression of the state, disorder or condition or at least one of its clinical or sub-clinical symptoms or signs.

As used herein, the term “tuberculosis” refers to the disease caused by the bacteria Mycobacterium tuberculosis (M.tb). A latent M.tb infections refers to when a subject is infected with M.tb but does not exhibit any signs or symptoms of tuberculosis and are not infectious. Latent M.tb infections can be diagnosed via a blood test or a tuberculin skin test. Active M.tb infection refers to when a subject exhibits signs and symptoms of the disease (tuberculosis) and is infectious.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, the terms “therapeutically effective amount”, “therapeutically effective dose” and “effective amount” refer to an amount of the compound and compositions which is sufficient to effect beneficial or desired results, that, when administered alone or in combination with an additional therapeutic agent to a cell, tissue, or subject, is effective to cause a measurable improvement in one or more symptoms related to the particular disease or medical condition. A therapeutically effective dose further refers to that amount of the compound sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount can also result in an improvement in a subjective measure in cases where subjective measures are used to assess disease severity.

### Phosphatidylserine-Binding Molecules and Associated Pathways

In accordance with one or more embodiments of the present application, phosphatidylserine-mediated pathways are utilized as an HDT target. Exposure of phosphatidylserine (PS) on the surface of tumor cells creates an immunosuppressive environment and its inhibition by anti-PS biomolecules (e.g., anti-PS antibodies) can promote a more immunoresponsive microenvironment by converting M2 macrophages, which are known to suppress immune responses, into the more beneficial M1 phenotype(31). In addition, anti-PS antibodies have been shown to neutralize HIV-1 infection by inducing the CCR5 cytokines from the macrophage(32). We have recently found that PS molecules exposed on activated immune cells function as checkpoint targets, and that masking of PS with particular anti-PS antibodies leads to increases in the number of antigen-activated memory T cells with effector functions, resulting in significant reduction in M.tb growth in human peripheral blood mononuclear cells (PBMCs) isolated from a latently TB infected individual. These results provide the rationale for developing appropriate PS antibodies as immunotherapeutics against latent TB coinfection.

Additionally, the present application displays the potential of utilizing PS as a checkpoint target on immune cells and harnesses the natural anti-PS antibody response to increase effector function in exhausted CD4 and CD8 memory T cells. Such a novel approach may have promise as a more efficient and targeted treatment for various diseases, particularly as potential adjunctive therapeutics to current antibiotic treatments for MDR and XDR tuberculosis. Additionally, the present methods have implications for understanding the role of antiphospholipid antibodies (aPLs) in autoimmune diseases, such as in lupus and rheumatoid arthritis, and can lead to the development of targeted therapies for these diseases as well. Furthermore, the present methods can aid in early diagnosis of latent infectious diseases using anti-PS antibody mediated improved interferon gamma release assay (IGRA) to differentiate the CD4 and CD8 response, allowing for timely treatment. The examples of the present application also shed light on the mechanisms of the innate antibody response and its role as natural adjuvants in enhancing the immune response to vaccines, which can lead to the development of more effective vaccination strategies for immunogens with low immunogenicity, e.g., cancer vaccines.

Anti-PS antibodies have been previously shown to have potential in controlling multiple viral infections by directly binding to the exposed PS on viral membrane and the infected cells(33, 34) or indirectly by inducing the CCR5 cytokines(32). However, their use as therapeutics has been limited due to their weak neutralizing activity. The present application focuses on the highly innovative approach to harness the specific functions of innate antibodies, which are poorly studied and largely ignored, due to their known polyreactivities and other issues. For instance, in one or more embodiments, the present application is based on the discovery of anti-phosphatidylserine antibodies having the potential to restore the effector functions of exhausted or antigen nonresponsive memory T cells by blocking the exposed PS on the surface of immune cells. The present application describes a significant development showing that such antibodies enhance the activation of antigen-specific memory T cells, and the clearance of TB infection in vitro. This research aims to investigate the novel and complex interactions between activating and inhibitory signals in the immune system, which are crucial for maintaining a balanced and effective immune response. Blocking the suppressive signal of PS on memory T cells, which appears to be associated with immune checkpoints has the potential to restore the effector functions of these cells upon exposure to antigens. The studies in the examples examine how manipulation of exposed PS on immune cells by pathogens can promote their growth and latency and can lead to a better understanding of the functions of innate antibody responses during infection. Harnessing these responses can further lead to the development of novel therapeutics that target changes in the host's immune components. Utilizing the host's own immune response can be a more effective strategy in overcoming pathogen-mediated immunosuppression, compared to synthetic small-molecules which would affect every cell, and have unanticipated side-effects.

Multiple pathogens have evolved distinct mechanisms to manipulate the host's immune response and promote their own survival and dormancy within cells. As a result, treatment strategies that are effective against one pathogen may not work as well against others. This is particularly true in cases of coinfection, where the use of one inhibitor may even enhance the infectivity of another pathogen (as evidenced by the relationship between PD1 inhibitors on HIV-1 viral load and M.tb pathogenicity). The higher prevalence of plasma PS antibodies in HIV-1 infected individuals suggests a potential link to increased T cells mediated inflammation and T cells exhaustion in these patients. In contrast, individual with latent TB exhibit no detectable plasma PS antibodies and harbor antigen-nonresponsive memory T cells. Characterizing the modulation of immune checkpoints markers by PS antibodies provides valuable insights into potential HDT combinations for treating HIV-TB coinfection. The present discovery that anti-PS antibodies induce an increase in TIM3 expression while reducing TIGIT levels on T cells represents a novel contribution to this field. This finding has significant therapeutic implications, particularly in pathological conditions characterized by elevated plasma anti-PS antibody titer, and could pave a way for innovative treatment strategies.

Apart from the development of novel HDTs against infectious diseases, the present research also provides a novel approach to evaluate the level of pathogen mediated immunosuppression of memory T cells, by quantifying the increase in the effector function of Ag-activated memory T cells in the presence of anti-PS antibody. In addition, the present discoveries, as exemplified in the following examples, offers a unique means of measuring the strength of innate immune response in individuals, allowing for the early prediction of a balanced T cell response or potential inflammation and tissue damage. In summary, the examples of the present application explore the diversity of natural antibodies that have the ability to bind directly to PS or indirectly through mediator proteins and have potential immunomodulatory functions.

In accordance with one or more embodiments, the present application provides a method of restoring effector function in antigen non-responsive or exhausted memory T cells. In the method, a therapeutically effective amount of phosphatidylserine (PS)-binding biomolecules is delivered to a one or more peripheral blood mononuclear cells (PBMCs) or whole blood of a subject in the presence of antigens specific to one or more diseases or neoantigens specific to cancer cells, wherein the PBMCs or whole blood comprise memory T cells that are exhausted or non-responsive to the antigens specific to the disease or the neoantigens specific to cancer cells. The delivery of the PS-binding biomolecules to the PBMCs or the whole blood restores effector function in the antigen-exhaust/nonresponsive or neoantigen-exhausted/nonresponsive memory T cells. In one or more embodiments, method can be performed in vitro, using a test tube or laboratory dish, for example, and a blood sample of the subject. In one or more embodiments, the method can be performed in vivo by administrating the PS-binding biomolecules to the subject via one of more routes of administration, including but not limited to oral, rectal, parenteral, intravenous, intramuscular, and subcutaneous. In one or more embodiments, the subject is co-infected with HIV and Mycobacterium tuberculosis (M.tb), and wherein the antigens are HIV and M.tb-specific antigens.

In one or more embodiments, the method can further include quantifying the increase in effector function of the memory T cell, e.g., by Flow Cytometry, ELISPOT, or IGRA assay. In one or more embodiments, the memory T cells are CD4+ memory T cells or CD8+ memory T cells. In one or more embodiments, the subject can be a mammalian subject, such as a human. In one or more embodiments, the subject (e.g., human) has previously taken a vaccine for the disease. In one or more embodiments, the disease is caused by the SARS-CoV2 virus or Mycobacterium tuberculosis. However, it should be understood that in one or more embodiments, the disease can be caused by one or more other pathogens (e.g., virus, bacterium, protozoan, prion, viroid, or fungus). In one or more embodiments, the disease can be a cancer or an autoimmune disease.

In one or more embodiments, the PS-binding biomolecules are antibodies, such as monoclonal antibodies (PS mAbs) or polyclonal antibodies. In one or more embodiments, the PS mAbs can be incorporated into the recommended concentration range of FDA approved therapeutic antibodies (100-200 mg/ml) (35) through in vivo optimization. In at least one embodiment, the PS-binding biomolecules are annexins. In one or more embodiments, the PS-binding biomolecules are synthetic small molecules, e.g., (18)F-dipicolylamine (DPA) or similar derivatives.

In one or more embodiments, a method of diagnosing a latent Mycobacterium tuberculosis (M.tb) infection in a subject is provided. In this method, a blood sample of a subject is obtained, where the blood sample comprises memory T cells. The blood sample of the subject is then assayed with PS-binding biomolecules in the presence of M.tb-specific antigens. In one or more embodiments, the assay is an Interferon Gamma Release Assay (IGRA) or an ELISPOT assay or QUANTIferon-TB GOLD Plus (QFT-Plus) assay or Flow cytometry. In one or more embodiments, the M.tb-specific antigens are early secretory antigen target-6 (ESAT6) and culture filtrate protein-10 (CFP-10). A level of IFNγ in the blood sample is then measured via the assay. Based the level of IFNγ in the assay it is then determined whether the subject has a latent M.tb infection. In one or embodiments, when utilizing the QUANTIferon-TB GOLD Plus (QFT-Plus) assay, a sample having IFNγ≥0.35 IU/ml and ≥25% of Nil is considered positive for the TB infection. In one or more embodiments, the assay is an Interferon Gamma Release Assay (IGRA) or an ELISPOT assay. In one or more embodiments, the memory T cells are CD4+ memory T cells or CD8+ memory T cells. In one or more embodiments, the PS-binding biomolecules are antibodies, such as monoclonal antibodies or polyclonal antibodies. In at least one embodiment, the PS-binding biomolecules are annexins. In one or more embodiments, the PS-binding biomolecules are synthetic small molecules.

In one or more embodiments, TIGIT and/or LAG3 inhibitors, alone or in combination, may be utilized to optimize CD3 activation for the reactivation of the latent reservoir of HIV-1 infected dormant CD4+ T cells in the presence of antiretroviral therapy, with the goal of achieving complete viral eradication in a HIV-1 infected subject.

In one or more embodiments, a method of enhancing CD4/CD8 memory T cell response in a subject with a latent Mycobacterium tuberculosis (M.tb) infection. In the method, a therapeutically effective amount of PS-binding molecules is administered in the presence of M.tb-specific antigens to the subject with the M.tb infection. In one or more embodiments, the PS-binding molecules can be PS-binding monoclonal antibodies (PS mAb), which can be utilized in the recommended concentration range of FDA approved therapeutic antibodies (100-200 mg/ml) through in vivo optimization. In one or more embodiments, the PS-binding biomolecules is administered to the subject via one of more routes of administration, including but not limited to oral, rectal, parenteral, intravenous, intramuscular, and subcutaneous. In one or more embodiments, the memory T cells are CD4+ memory T cells or CD8+ memory T cells. In one or more embodiments, the PS-binding biomolecules are antibodies, such as monoclonal antibodies or polyclonal antibodies. In at least one embodiment, the PS-binding biomolecules are annexins. In one or more embodiments, the PS-binding biomolecules are synthetic small molecules. In one or more embodiments, the subject is co-infected with M.tb and HIV-1.

In one or more embodiments, a method of diagnosing whether a latent Mycobacterium tuberculosis (M.tb) infection will progress to active M.tb disease in a subject is provided. In the method, a blood sample of a subject previously diagnosed with latent M.tb is obtained, where the blood sample comprises memory T cells. The obtained blood sample of the subject is then assayed with PS-binding biomolecules in the presence of CD4 and CD8 antigens. In one or more embodiments, the assay is a QuantiFERON^{(R)} TB Gold assay. Similar assays can be developed for many other pathogens by stimulating the T cells with pathogen-specific antigens in the presence of anti-PS mAbs to determine the latent infection or to measure the T cell immunity. In one or more embodiments, ELISPOT or IGRA assays can be utilized, for example.

In one or more embodiments, the level of IFNγ in the blood sample is then measured via the assay (e.g., IGRA assay). It is then determined, based on the measured level of IFNγ induced by CD4+ and CD8+ T cell, whether the latent M.tb infection will progress to active M.tb disease. In one or more embodiments, the PS-binding biomolecules are antibodies, such as monoclonal antibodies or polyclonal antibodies. In at least one embodiment, the PS-binding biomolecules are annexins. In one or more embodiments, the PS-binding biomolecules are synthetic small molecules. In one or more embodiments, the subject is co-infected with M.tb and HIV-1.

In one or more embodiments, a method of treating one or more active or latent infections in a subject is provided. In the method, a therapeutically effective amount of phosphatidylserine (PS)-binding biomolecules is administered to the subject in the presence of antigens specific to one or more diseases that cause the one or more active or latent infections. The administration of the PS-binding biomolecules to the subject in the presence of T cell antigens restores effector function in memory T cells of the subject that are exhausted or non-responsive to the disease-specific antigens. In one or more embodiments, the active or latent infection is caused by HIV or Mycobacterium tuberculosis (M.tb). In at least one embodiment, the subject is co-infected by HIV and M.tb. In one or more embodiments, the memory T cells are CD4+ memory T cells or CD8+ memory T cells. In one or more embodiments, the PS-binding biomolecules are antibodies, such as monoclonal antibodies or polyclonal antibodies. In at least one embodiment, the PS-binding biomolecules are annexins. In one or more embodiments, the PS-binding biomolecules are synthetic small molecules.

In certain embodiments of the present methods, the M.tb infections can be multidrug-resistant (MDR) or extensively drug-resistant tuberculosis. In one or more embodiments, the therapeutically effective amount of the antibodies is approximately 100-200 mg/ml.

In one or more embodiments, an overproduction of PS-binding biomolecules in a bacterial or viral infections or in an autoimmune disease can be analyzed to determine their ability to induce memory T cells mediated inflammation.

In one or more embodiments, the PS-binding biomolecules can enhance the memory T cells immunity against pathogens or the cancer cells by being utilized in a vaccine containing pathogen-specific antigens or cancer cell-specific neoantigens.

In one or more embodiments, the PS-binding biomolecules can be utilized as common vaccine antigens and antiretroviral drugs to activate latent CD4 T cells reservoirs of dormant T cells infected with HIV-1, ultimately eradicating the virus to cure the patient.

In one or more embodiments, a method of restoring effector function in antigen non-responsive or exhausted memory T cells of a subject is provided. In the method, a blood sample is obtained, and then a therapeutically effective amount of phosphatidylserine (PS)-binding biomolecules is delivered to the blood sample in the presence of antigens specific to one or more diseases or neoantigens specific to cancer cells. The blood sample comprising the phosphatidylserine (PS)-binding biomolecules in the presence of the antigens or neoantigens is then transfused to the subject. The transfused blood sample restores effector function in memory T cells of the subject that are exhausted or non-responsive to the antigens specific to one or more diseases or the neoantigens specific to cancer cells. In one or more embodiments, the antigens are M.tb-specific antigens or HIV-1-specific antigens. In one or more embodiments, the antigens are SARS-CoV2-specific antigens. In one or more embodiments, the PS-binding biomolecules are antibodies. In one or more embodiments, the antibodies are monoclonal antibodies or polyclonal antibodies. In one or more embodiments, the PS-binding biomolecules are annexins. In one or more embodiments, the PS-binding biomolecules are synthetic small molecules.

In one or more embodiments, PS targeting antibodies modulate the checkpoints receptors on the T cells in the absence of T cells antigens. In one or more embodiments, PS targeting antibodies makes the T cells less resistant to antigen activation by reducing the expression of inhibitory receptors.

In one or more further embodiments, PS-binding biomolecules (e.g., PS antibodies) can be used as an adjuvant with a vaccine immunogens to induce an effective T cells response.

In at least one embodiment, the PS-binding biomolecules are antibodies. In a further aspect, the antibodies are monoclonal antibodies. In a further aspect, the antibodies are polyclonal antibodies. In another aspect, the PS-binding biomolecules are annexins. In another aspect, the PS-binding biomolecules are synthetic small molecules.

In one or more embodiments, a method of treating one or more active infections in a subject is provided. In the method, a therapeutically effective amount of soluble TIGIT alone or in combination with TIM3 is administered to the subject with high titer of plasma PS antibodies to resolve inflammation and T cells exhaustion in the subject. In at least one embodiment, the subject is co-infected by HIV and M.tb or with autoimmune disease.

In at least one embodiment, TIM3 inhibitors or the soluble TIGIT or the combination of both can be evaluated at a dose ranging from approximately 20 mg to 1600 mg administered intravenously every 1 to 4 weeks.

In at least one embodiment, HIV-1 infected subjects maintain a high-titer of PS targeting plasma antibodies. In at least one embodiment, high-titer of PS targeting plasma antibodies leads to T cells mediated inflammation and T cells exhaustion.

In at least one embodiment, inflammation followed by T cells exhaustion induced by PS targeting antibodies are mediated by increased TIM3 and decreased TIGIT receptors on T cells.

In at least one embodiment, inhibitors of TIM3 or soluble TIGIT or their combination can be used as host directed therapeutics (HDT) to resolve the PS targeting antibodies mediated inflammation.

In one or more embodiment, a method of using PS antibody as adjuvants with vaccine immunogens to induce an effective T cell response is provided.

The above aspects and other aspects of the present methods can be further understood through the following examples.

### EXAMPLES

Biological membranes require an asymmetric distribution of lipids across the bilayer in order to preserve membrane potential and carry out crucial biochemical functions. This involves predominantly choline-containing phospholipids (PC and SM) being present in the outer leaflet, while amino-phospholipids (PS, PE, and PI) are primarily located in the inner leaflet. In normal conditions, PS asymmetry is controlled) by three types of enzymes, but their balance can be disrupted during apoptotic processes and cellular stress. Flippases and floppases use ATP to transfer phospholipids between the outer and inner surfaces, while scramblases regulate PS topology by causing PS to accumulate on the external side of the membrane, thereby disrupting asymmetry when activated. Despite this, the precise mechanisms of scramblase activity are not fully understood. In physiological states, lipid transporters are tightly regulated in native cells, but in pathological situations, disruption of membrane bilayer asymmetry has been linked to compromised membrane function during cellular stress. For instance, in caspase-mediated apoptosis, the activation of the Xkr8 (Ced8) scramblase leads to externalization of PS, which marks cells for recognition and degradation through phagolysosomes (a process known as efferocytosis). Similarly, in cells experiencing reactive oxygen species (ROS) and hypoxia, PS is externalized through the function of TMEM16, which is essential for platelet activation and nucleation of clotting factors on the surface of endothelial cells. Phosphatidylserine (PS) re-localization to the external surface of the plasma membrane during caspase-mediated apoptosis also plays a role in PS-mediated phagocytosis of apoptotic cells.

PS expression has also been observed on the surface of viable cells, including activated platelets, monocytes, mature macrophages, activated B and T cells, dendritic cells, tumor vasculature, tumor cells, and exosomes from tumors. In these viable cells, the externalization of PS does not result in phagocytosis, as phagocytes can differentiate between PS on living and apoptotic cells. The purpose underlying the PS externalization on live activated immune cells is not yet fully understood. Multiple cell surface receptors and endogenous serum proteins, known as PS receptors, have been identified, including members of the TAM and TIM family of cell surface receptors (Tyro3, Axl, Mertk, TIM1, TIM3, and TIM4), as well as serum proteins such as C1q, MFG-E8, Gas6, Pros1, coagulation factors V and VII, phospholipase A2, scavenger receptors class B Type-1 (SR-B1), phospholipid transfer protein (PLTP), lipoprotein lipase (LPL), annexin and apolipoproteins. Similarly, in viable cells exhibiting elevated PS levels, there is no phagocytosis as immune cells are able to distinguish between living and apoptotic cells that express PS. The purpose of PS expression on live activated immune cells remains unclear. Multiple cell surface receptors and serum proteins, also known as PS receptors, have been identified, including TAM and TIM family members (Tyro3, Axl, Mertk, TIM1, TIM3, and TIM4), as well as serum proteins such as C1q, MFG-E8, Gas6, Pros1, coagulation factors V and VII, phospholipase A2, scavenger receptor class B type-1 (SR-B1), phospholipid transfer protein (PLTP), lipoprotein lipase (LPL), annexins, and apolipoproteins. PS receptors have shown to play a role in clearing apoptotic cells, as has been observed with the effects of TIM receptors on the early stage of phagocytosis, where TIM receptor knockout in mice leads to defects in the clearance of apoptotic cells. This was also observed with the blocking of TIM receptors by antibodies, which resulted in the development of autoreactive B and T cells. Interestingly, low levels of phosphatidylserine-targeting antibodies are present in healthy individuals, with levels increasing upon infection and subsiding once the infection is resolved, without causing any autoimmune-related pathology. In fact, these antibodies have shown beneficial properties, such as the ability to inhibit viruses or exhibit anti-tumor effects, without causing any autoimmune-related pathology. However, the direct mechanism by which phospholipid antibodies can be protective or pathogenic remains unknown.

In the following studies, blood and/or PBMCs from individuals infected with COVID-19 or TB were used to discover that the anti-PS monoclonal antibody 11.31 can restore effector function in antigen non-responsive memory T cells. The sequences (heavy chain [HC] and light chain [LC]) for the anti-PS monoclonal antibody 11.31 are shown below, and are described in U.S. Patent Application Publication No. 2011/0318360 (U.S. patent application Ser. No. 12/737,987).

11.31 HC
QVQLQESGPGLVKPSGTLSLTCAVSGGSISSSNWWSWVRQPPGKGLEWIG
EIYHISGSTNYNPSLKSRYTISVDKSKNQFSLKLSSYTAADTAVYYCARS
RERSWLVKRRYVYFDYWGQGTLYTVSSASTKGPSYFPLAPSSKSTSGGTA
ALGCLVKDYFPEPVTVSWNSGALTSGVHIFPAVLQSSGLYSLSSVVTVPS
SSIGTQTYICNYNHKPSNTKVDKKVEPKSCDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELIKNQVSLTCLVKGFYPSDIAVEWESNGQPENN
YKTIPPVLDSDGSFFLYSKLTVDKSRWQQGNVFSCSVMHEALHNHYTQKS
LSLSPGK
11.31 LC
SSELSQDPAVSVALGQTVRITCQGDSLRSYYASWYQQKPGQAPVLVIYGK
NNRFSGIPDRESGSSSGNTASLTITGAQAEDEADYYCNSRDSSGNVVFGG
GTKVTVLGQPKAAPSVTLFPPSSEELQANKATLVCLISDFYPGAVTVAWK
ADSSPVKAGVETTIPSKQSNNKYAASSYLSLTPEQWKSHRSYSCQVTHEG
STVEKTVAPTECS

This example indicates that anti-PS antibodies can be utilized as a therapeutic approach to treat various active or latent infections in immunocompromised individuals or cancer patients. The results also demonstrated that the anti-PS antibody-mediated restoration of memory T cells, in the presence of respective antigens, greatly improved the performance of the interferon-gamma release assay. This assay maybe used as an ultrasensitive diagnostic tool for latent infections and to differentiate between CD4 and CD8 memory T cell responses to predict disease activation. Moreover, the following examples show that the improved effector function of memory T cells resulted in better control of Mycobacterium tuberculosis (M.tb) growth when PBMCs from latent TB patients were infected in the presence of the PS monoclonal antibody 11.31. In addition, the plasma of 82 COVID-19 convalescent individuals were screened and a higher level of anti-PS IgG in those with more inflammatory symptoms was found. Four selected COVID-19 convalescent plasma samples with high levels of anti-PS antibodies were then tested for their ability to induce effector function in CD3/28 activated T cells and their capability to restore effector function in antigen-activated memory T cells. The results confirmed that these plasma samples had similar effects on T cells as the PS monoclonal antibody 11.31 observed earlier. This supports the idea that the treatment of autoimmune diseases with high levels of phospholipid antibodies, or the use of immunosuppressive agents for conditions such as anti-phospholipid antibody syndrome, can be achieved by using an anti-CD3 antibody to suppress the effector function of T cells. The findings also provide a novel method to measure inflammation mediated by phospholipid antibodies or phospholipid-binding plasma proteins, which can be used to monitor disease progression. Additionally, it was confirmed that phospholipid antibodies induced during infection can similarly boost effector function of memory T cells using Protein-G column-purified polyclonal plasma antibodies from four selected COVID-19 convalescent individuals with differential anti-PS antibody titers. The findings provide insight into the mechanism behind anti-phospholipid antibody function and highlight their completely novel diagnostic and therapeutic approach.

### Methods

### Flow Cytometry Analysis of CD3/28 and Antigen Activated T Cells

Flow cytometry analysis was performed to evaluate the impact of PS monoclonal antibodies or purified polyclonal plasma antibodies on SARS-CoV2 antigen-activated memory T cells isolated from individuals who have been vaccinated for COVID-19. A total of 40 ml of blood was collected from a vaccinated blood donor and subjected to centrifugation to obtain plasma. The plasma was then aliquoted into vials and stored at −80° C. The remaining blood cells were resuspended in 1X PBS and subjected to Ficoll density gradient centrifugation to separate the peripheral blood mononuclear cells (PBMCs). The counted PBMCs were stored at a concentration of 10×10⁶ cells per cryovial in freezing solution (10% DMSO and 90% FBS) and placed in liquid nitrogen for future use. For T cell activation assays, the PBMCs were thawed and plated in 96-well plates at a density of 250,000 cells per well in RPMI 1640 medium supplemented with 10% heat-inactivated FBS and 2% L-glutamine. The cells were then activated with a combination of CD3 and CD28/49d Fastimmune antibodies, along with PHA-L and a defined peptide pool for SARS-CoV2 antigen (purchased from Mabtech) for a duration of 10 hours. GolgiPlug was added during the activation process to inhibit cytokine secretion, and the staining protocol provided by BD Biosciences was followed. Prior to staining for cell surface markers, the cells were treated with Fc block reagent and DAPI at room temperature. The flow cytometric analysis included the use of anti-CD3-FITC, anti-IFN-g-AF594, and anti-TNF-a-AF647 antibodies. A total of 100,000 PBMCs were acquired and gated to determine the percentage of CD3+ T cells expressing intracellular cytokines IFN-g and TNF-a, as described previously (36). Experiments were performed in duplicate and the data were normalized to the stimulated controls. Additionally, protein-G purified total antibodies from plasma, identified through PS binding ELISA, were also tested for their impact on CD3/28 and SARS-CoV2 antigen-activated T cells using a similar method.

### ELISPOT Assay to Analyze the CD3/28 and Antigen Activated T Cells

An ELISPOT Assay was utilized to assess the impact of PS mAB 11.31 on antigen-stimulated memory T cells isolated from solid organ transplant patients. Blood samples (20 ml) were obtained from solid organ transplant recipients who had received the COVID-19 vaccine and had been on immunosuppressive medication for at least two years. Plasma and PBMCs were separated following the methods outlined previously. The Human IFN-γ SARS-CoV2 ELISpotPLUS kit (product code: 3420-4AST-P1-1, Mabtech) was used to perform the assay, with all steps conducted in a sterile biosafety cabinet. Cells were stimulated with five different stimuli, including S1, S2, SNMO peptide pools for SARS-CoV2, and CD3/28 antibodies (Abs) and Phytohemagglutinin (PHA) as a positive control. Wells without stimuli served as negative controls. Freshly isolated PBMCs from solid organ transplant recipients (SOTRs) were suspended in 10% FBS-AMEM media at a concentration of 2.5 million/ml. 100 ul of cells-suspension were added to each well and the plate was incubated in a humidified incubator at 37° C. with 5% CO2 for 15 hours. The plate was then washed 4 times with 1× PBS before incubation with the detector antibody 7-B6-1-biotin at a concentration of 1ug/ml in 0.5% FBS-1× PBS for 2 hours at room temperature. After the incubation was over plates were washed and streptavidin-ALP (1:1000) was added to each well in PBS-0.5% FBS and plates were incubated for 1 hour at room temperature. Next, a BCIP/NBT-plus substrate solution was filtered using a 0.45 ul filter and 100 ul was added to each well of the finally washed plate. The plate was then monitored for spot formation and once spots emerged, milliQ water was added to stop the reaction. After allowing the plate to dry, spots were counted using an ELISpot reader. The number of spots in wells treated with the same stimulants with and without PS mAb were compared to evaluate the effect of PS mAb on CD3/28, PHA and antigen activated T cells.

### QUANTIferon-TB GOLD Plus (QFT-Plus) Assay

The QuantiFERON-TB GOLD Plus kit (Qiagen, USA) was utilized for conducting the interferon (IFN)-γ release assay (IGRA) on blood samples with known tuberculosis (TB) diagnosis and healthy control. This kit can detect secreted IFN-y from memory T cells in response to two TB-specific antigens, early secretory antigen target-6 (ESAT6) and culture filtrate protein-10 (CFP-10), aiding in the diagnosis of latent or active TB. The assay was carried out following the provided protocol. A blood sample was assigned two kit packets, each containing four tubes labeled as TB1, TB2, NIL, and PHA. These tubes were then labeled with the corresponding sample ID and either No PS mAb or Plus PS mAb. To each QuantiFERON-TB GOLD Plus collection tube, 1 mL of freshly collected blood was added, followed by 100 μl of 1×PBS or 1 mg/ml PS mAb (11.31) depending on the tube label. The tubes were gently shaken to ensure complete dissolution of the antigens on the tube walls and incubated at 37° C. (±1° C.) for 16-24 hours. After incubation, the tubes were centrifuged at 3000 g for 15 minutes, and the plasma was collected in labeled 1 mL tubes. The collected plasma was either stored at −80° C. for future analysis or immediately tested using the IFNγ Enzyme-Linked Immunosorbent Assay (ELISA) kit provided with the QuantiFERON-TB GOLD Plus kit. The IFNγ concentrations in IU/ml were determined using a standard curve prepared with the kit's recombinant IFNγ protein. The results were then interpreted according to the kit's guidelines to determine a positive or negative response.

### M.tb Infection of Human PBMCs

PBMCs from individuals with latent tuberculosis infection and healthy individuals were assessed for their ability to control Mycobacterium tuberculosis (M.tb) infection and growth, both with and without the presence of anti-PS monoclonal antibody (mAb). Frozen PBMC samples were retrieved from liquid nitrogen storage, thawed, and viable cells were counted to distribute 0.5 million cells into each well of a 24-well tissue culture plate. Plates were prepared in triplicate, with each plate containing three replicates of PS mAb untreated and treated cells infected with clinical strain H37Rv. To account for the approximately 20% monocytes in PBMCs, a maximum MOI of 10:1 was used, by adding 1 million bacteria to 0.5 million PBMCs in each well. At 3, 72, and 120 hours, cells from each well were centrifuged, and the resulting cell pellet was lysed in a solution containing 0.05% Triton X-100. The lysate was serially diluted in a solution of 0.05% Tween-80 in PBS and plated on 7H11 Agar plates. After three weeks, colony formation units (CFUs) were counted to evaluate the effect of anti-PS mAb on bacterial growth in culture.

### PS and CL Binding ELISA

The brain-derived L-phosphatidylserine and heart-derived cardiolipin were procured from Avanti Polar Lipids (Cat: 840032P and 840012) and dissolved in methanol. Ninety-six well ELISA plates (Greiner bio-one) were coated with 50 μL of PS or CL at a concentration of 12.5 μg/mL. The plates were left in a sterile fume hood until the solvent evaporated. They were then blocked with 5% BSA in PBS overnight at 4° C. After being washed with lipid-wash buffer (10 mM HEPES, 150 mM NaCl, 2.5 mM CaCl2), the plates were incubated with diluted human plasma or the purified plasma antibodies for two hours at 37° C. Subsequently, 50 μL of alkaline phosphatase-conjugated anti-human (H+L) secondary antibodies were added to the plates and incubated for one hour at 37° C. After washing with corresponding buffers, the plates were treated with AP substrate (Sigma S0942) dissolved in diethanolamine buffer and the absorbance was measured at 405 nm. Each sample was tested in duplicate and the average absorbance was considered the binding antibody signal. Samples with values above 3× OD(405) with BSA were deemed positive. The final binding results were obtained after subtracting the background value (3× OD(405)-BSA).

### Protein-G Purification of Plasma Antibodies

To purify plasma antibodies previously shown to strongly bind to PS by ELISA, Recombinant Protein G beads (Invitrogen Catalogue no. 101243) were employed. The beads were incubated with diluted plasma samples ( 1/10 dilution in 1× PBS) in a 2 ml column, which was placed in the cold room overnight on a rotator. The collected flow through was then assessed for the proportion of unabsorbed antibodies using ELISA. Following PBS washing, the bound antibodies were eluted with 0.1M glycine-HCL (pH 2) and concentrated to 1× PBS using 50 kDa molecular weight concentrators. The resulting purified antibodies were further filtered through a 0.22 micron centrifuge tube filter and evaluated for their binding ability to PS as well as their impact on CD3/28 and SARS-CoV2 antigen-stimulated T cells.

### Frequency of IFNγ Positive CD4 and CD8 T Cells Subpopulation Upon Antigen and CD3 Activation in the Presence and Absence of PS Antibody

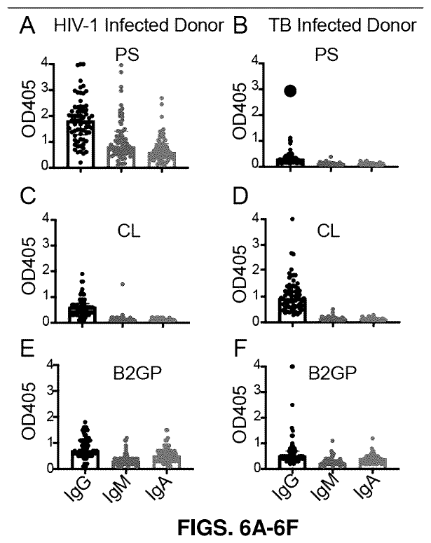

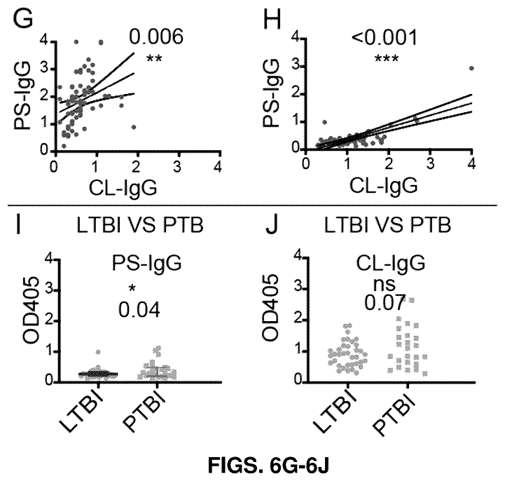

To further understand the mechanism of phosphatidylserine (PS)-targeting antibodies mediated increase in the number of functional T cells activated by the antigens, while simultaneously reducing the number of functional T cell activated by anti-CD3/28 antibodies, PS expression on exhausted T cells and the potential role of cell-surface PS as a checkpoint regulator were assessed. Freshly isolated human peripheral blood mononuclear cells (PBMCs) were activated with T cell vaccine antigens and CD3/28 antibodies, both with and without the PS-targeting monoclonal antibody (mAb) 11.31 and an isotype control. CD4 and CD8 T cells were categorized into four subpopulations based on the differential expression of CD45RA, CCR7, and CD45RO: naïve-like, central memory, effector memory, and terminally differentiated effector memory T cells (see FIGS. 6A-6B).

### Results

### Anti-PS Antibodies Restore the Effector Function of Antigen Non-Responsive Memory T Cells in Human PBMCs Treated With T cell Antigens

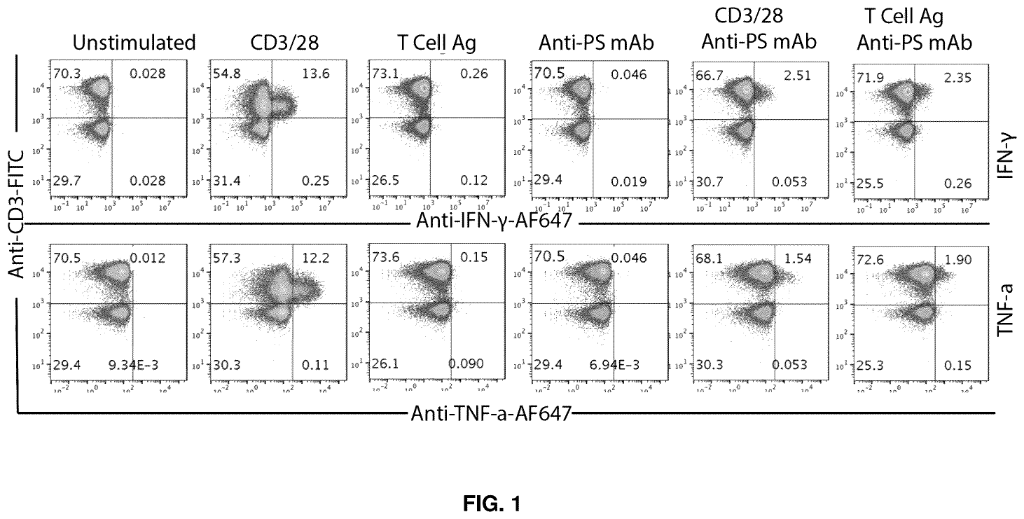

In order to gain a better understanding of exposed PS on activated immune cells, the impact of blocking the exposed PS on CD3/28 activated T cells by anti-PS antibodies was determined. The findings revealed an immunosuppressive effect of direct PS binder mAb, 11.31 on CD3/28 activated immune cells while indirect PS targeting mAb, BAVI had no such effect (36). However, since CD3/28 activation may not be a completely accurate representation of antigen-activated T cells, we delved further and examined the effects of anti-PS monoclonal antibodies on memory T cells activated by antigens (Ag). These tests were conducted on PBMCs isolated from healthy blood donors with a known history of receiving common annual vaccinations (i.e., COVID-19 and Influenza vaccine). Interestingly, it was found that the T cells activation in the presence of both the vaccine Ag and anti-PS mAb, 11.31 resulted in a 10-fold increase in the number of memory T cells positive for intracellular IFNγ⁺ (Upper panel of FIG. 1) and TNFα⁺ (Lower panel of FIG. 1), compared to Ag activation alone. It was further confirmed the findings of decreased IFNγ+ and TNFα+ in CD3/28 activated T cells when exposed to anti-PS antibodies. Additionally, it was found that overnight stimulation did not change the total CD3 T cell counts between the anti-PS antibodies treated and untreated groups, suggesting that the decrease in IFNγ+ and TNFα+ CD3/28-activated T cells may be due to anergization rather than decreased cell numbers. In contrast, an increase in functional memory T cells when exposed to anti-PS antibodies during peptide antigen activation was observed, which suggests that the PS antibody can help the exhausted memory T cells to resume the effector function. Furthermore, the use of recombinant AnxV protein and indirectly PS-binding anti-β2GP mAb also resulted in an increase in IFNγ+ and TNFα+ memory T cells (results not shown).

### Anti-PS mAb Rejuvenate the Memory T Cells Effector Function in PBMCs Exposed to T Cells Immunosuppressive Drugs

Decreased effector T cell function may increase the risk of developing opportunistic infections and tumor. Pathogens have the ability to manipulate and suppress immune cells, creating a favorable environment for their own growth similar to the immune-suppressing microenvironment found in tumors. In order for host directed immunotherapy to be effective against cancer and deadly pathogens, it is essential to have T cells that are functional and specific to the tumor and pathogen specific antigens. A potential approach for achieving this is through neoantigen-directed therapy, which involves targeting the body's immune response towards tumor-specific antigens in order to eliminate cancer cells. Similarly, to fight the MDR TB infection in HIV-1 infected patients needs an adjunctive HDT that can boost their functional memory T cell response while they are coinfected and immunocompromised. The aim was to understand if the anti-PS mAb can be effective in restoring the effector memory T cell response in individuals whose T cell response is significantly compromised.

To replicate the compromised T cell immune response, peripheral blood mononuclear cells (PBMCs) were utilized from a solid organ transplant donor who had been receiving immunosuppressive drug regimens for at least two years, and who had also been vaccinated and infected with COVID-19. The experimental approach involved using their PBMCs in a Human IFN-γ SARS-CoV2 ELISpotPLUS kit (Cat #3420-4AST-P1-1, MABTECH) to assess the effect of anti-PS antibody mediated increase in the number of spots in the presence of T cell COVID-19 antigens (SARS-CoV2 S1 scanning pool-1 and pool-2, SARS-CoV2 S2 N defined peptide pool, and SARS-CoV2 S N M O defined peptide pool), as well as positive controls (CD3/28 and PHA). The guidelines provided by the kit to perform the assay were followed.

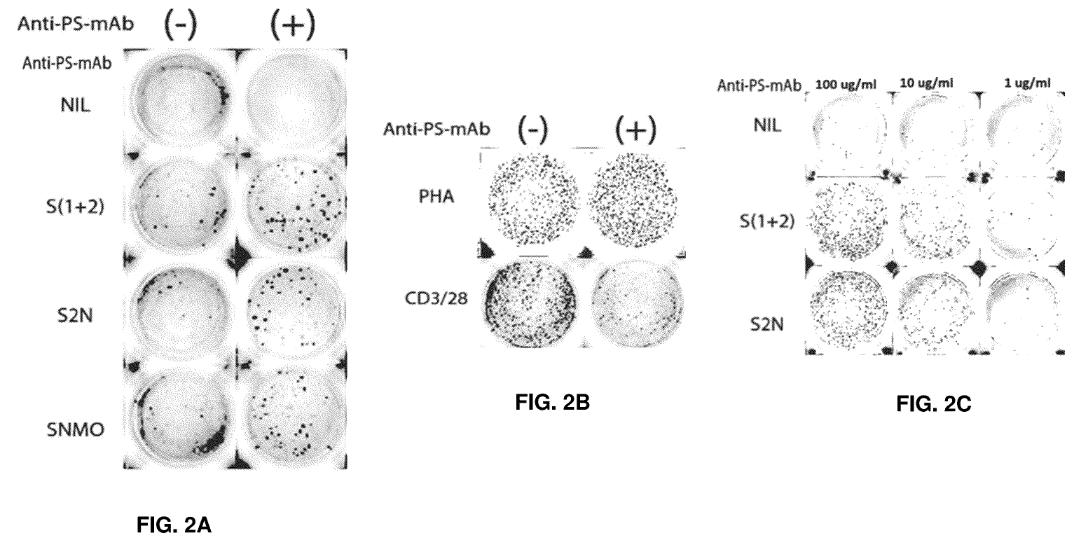

As expected, the PS antibody resulted in a significant increase in the number of IFNγ-secreting cell spots in the presence of SARS-CoV2 antigen pools (FIG. 2A), while a moderate increase in PHA-activated T cells and a significant decrease in the number of spots with CD3/28-activated T cells were observed (FIG. 2B-C). Furthermore, the PS antibody had a concentration-dependent effect, with the highest number of spots observed at 100 μg, followed by 10 μg and 1 μg/ml. The results suggest that anti-PS antibodies can restore effector functions in antigen nonresponsive memory T cells in the presence of antigens and show promise as host-directed therapeutics against pathogens or tumors which develops immunosuppressive conditions by regulating the host memory T cell response. The results further suggest that a combination of PS-mAb with pathogens specific T cell antigens or the neoantigens can significantly improve the functional memory T cells and can be used as HDTs to boost the T cells immunity against pathogens and cancer. It further suggests the adjuvant use of PS antibody with vaccine immunogen and neoantigens to induce strong memory T cell response.

### PS Targeting Antibodies Increases the Sensitivity of Existing Diagnostic Assay for Latent TB

Tuberculosis (TB) is second leading cause of death after COVID-19 from a single infectious disease worldwide. Latent TB infection (LTBI) is defined as a state of persistent immune response to stimulation by M.TB antigens with no evidence of clinically manifest active TB. Individuals with LTBI have a 5-10% lifetime risk of progressing to active TB. Individuals with LTBI represent a reservoir of infection and there remains to be significant uncertainty regarding the progression to active disease. However, the existing FDA approved assay has limited data available on the use of interferon gamma release assay (IGRA) in children less than 5 years, immunocompromised individuals or those recently exposed to TB. Two IGRAs that have been approved by the U.S. Food and Drug Administration (FDA) are commercially available in the U.S., (1) T-SPOT®.TB test (T-Spot) and (2) QuantiFERON®-TB Gold In-Tube test (QFT-GIT). T-SPOT®.TB test (T-Spot) assay is based on ELISPOT assay, and as shown earlier, PS antibody in ELISPOT assay significantly increase the sensitivity of the assay by restoring the effector function in antigen non-responsive memory T cells (FIG. 2A comparison between the ELISPOT assay done for the SARS-CoV2 specific memory T cells in the absence (−) and presence (+) of anti-PS mAb.

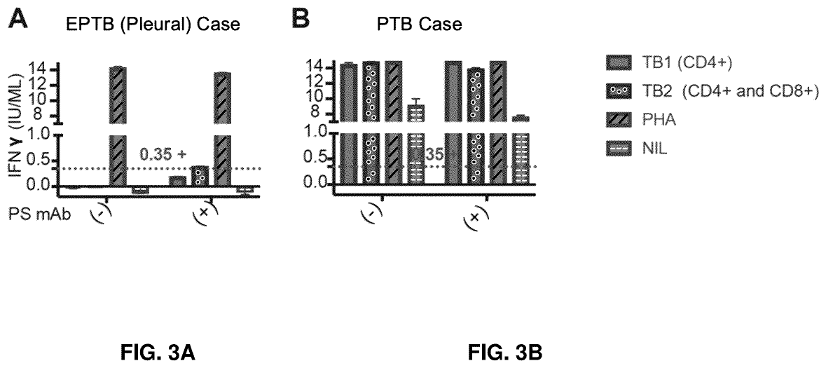

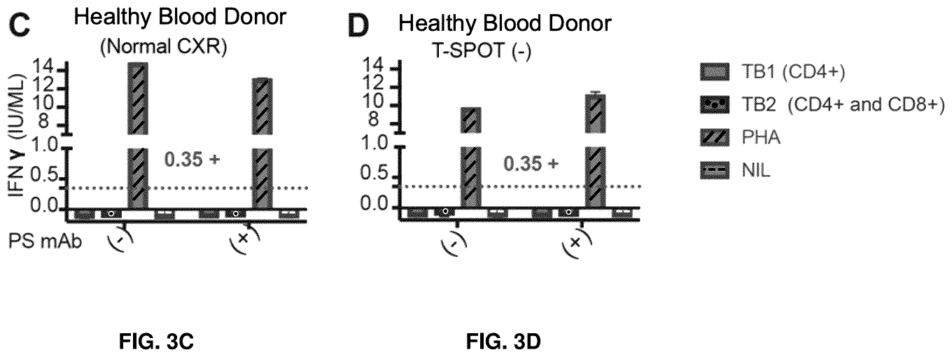

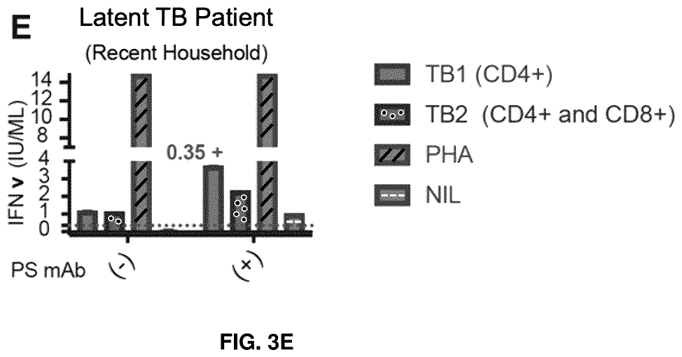

To further confirm that PS antibody can make the FDA approved IGRA assay for TB more sensitive, further investigations were conducted to learn if anti-PS mAb can increase the level of secreted cytokines by CD4 and CD8 memory T cells in response to CD4/CD8 Ags from the patient PBMCs diagnosed with latent TB. To do so, the FDA-approved QuantiFERON-TB GOLD Plus kit (Qiagen) was utilized to assess T cell activation in blood samples from five donors, including those with latent TB infection (LTBI), extrapulmonary TB (EPTB), pulmonary TB (PTB), and healthy control. These samples were stimulated with CD4 and CD4/CD8-derived peptide antigens from ESAT-6, CFP10 and CFP 7.7 proteins of Mycobacterium tuberculosis (M.tb), provided within the kit. Blood samples were assayed with and without the presence of anti-PS antibody, and the levels of secreted interferon-gamma (IFN-γ) were measured using an enzyme-linked immunosorbent assay (ELISA) as an indicator of memory T cell activation. A serial dilution of recombinant human IFN-y protein from the kit was used to generate a standard curve. Plasma samples were collected from four tubes with PS mAb and four tubes without PS mAb representing TB1-CD4, TB2-CD4/CD8, phytohaemagglutinin [PHA], No Ag [NIL], from each blood donor. Duplicate undiluted plasma samples were tested to determine the levels of IFN-γ in international units per milliliter (IU/ml). The results were interpreted using the algorithm shown in Table 1. The findings demonstrated a significant increase in IFN-γ levels when memory T cells were stimulated in the presence of PS mAb compared to antigen alone. No difference in IFN-γ levels was observed between CD4 and CD4/CD8 antigen tubes in the absence of PS mAb. However, the addition of PS mAb allowed for differentiation between CD4 and CD4/CD8 responses. Interestingly, a household contact of a PTB patient displayed a significantly higher CD4 response compared to CD4/CD8 mixed antigen stimulation in the presence of PS mAb (see FIG. 3E). In contrast, in the EPTB patient, the CD4/CD8 mix antigen elicited a higher response than CD4 in the presence of PS mAb (see FIG. 3A). No effect of PS mAb was observed in samples from two blood donors—one with a normal chest X-ray and tested negative in T-SPOT test, and the other with suspected respiratory tract infection but negative with T-SPOT TB test (see FIGS. 3C-D). The single donor diagnosed with PTB displayed high levels of IFN-y in all tubes, including the NIL tube with and without anti-PS mAb (see FIG. 3B). The results indicate that the addition of PS mAb can not only enhance the CD4/CD8 response, but also differentiate between CD4 and CD8 responses, providing a better understanding of disease progression from latent to active TB.

TABLE 1
Interpretation Guidelines for QFT-Gold Plus Results

| | | | Mitogen | | |
|---|---|---|---|---|---|
| Nil | TB1 minus | TB2 minus | minus Nil | QFT-Plus | Result |
| (IU/ml) | Nil (IU/ml) | Nil (IU/ml) | (IU/ml) | Result | Interpretation |
| ≤8.0 | ≥0.35 and | Any | Any | Positive | M. tuberculosis |
| | ≥25% of Nil | | | | infection likely |
| | Any | ≥0.35 and | | | |
| | | ≥25% of Nil | | | |
| | <0.35 or | <0.35 or | ≥0.50 | Negative | M. tuberculosis |
| | ≥0.35 and | ≥0.35 and | | | infection NOT |
| | <25% of Nil | <25% of Nil | | | likely |
| | <0.35 or | <0.35 or | ≤0.50 | Indeterminate | Likelihood of |
| | ≥0.35 and | ≥0.35 and | | | M. tuberculosis |
| | <25% of Nil | <25% of Nil | | | |

| | | |
|---|---|---|
| >8.0 | Any | Infection |
| | | cannot be |
| | | determined |

### Anti-PS mAb Significantly Reduces the Growth of M.tb Clinical Strain H37RV in PBMCs From Latent TB

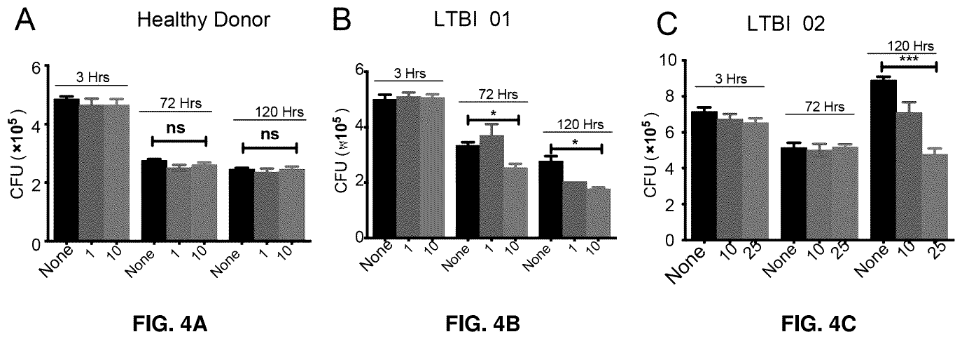

Previous data has demonstrated that the addition of anti-PS monoclonal antibody (mAb) to LTBI PBMCs stimulated with antigen increases the number of effector memory T cells and results in significantly higher levels of proinflammatory cytokines compared to antigen stimulation alone (FIG. 3). Based on these findings, the aim was to determine if anti-PS antibodies could provide protection against M.tb growth in LTBI PBMCs. PBMCs from three individuals were tested in this assay; one control PBMCs were obtained from blood bags through the blood bank, while two test PBMCs were isolated from LTBI diagnosed blood donors. M.tb clinical strain H37Rv was used to infect the PBMC at 1:2 MOI. As expected, the addition of PS antibody showed a protective effect against M.tb growth in both LTBI PBMCs, and no protective effect was observed in the control PBMCs from blood donor (FIG. 4A). The two LTBI PBMCs showed varying levels of protection, with one showing protection at 72 hours at a concentration of 10 ug/ml and at 120 hours for both 1 and 10 ug/ml (FIG. 4B). The second LTBI PBMC only showed protection at the 120-hour time point (FIG. 4C). A common observation was the dose-dependent effect and no protection observed at 3 hours post-infection, as Ag presentation may occur after multiple cycles of intracellular multiplication necessary for memory T cell receptor interaction and PS mAb-induced protection by reactivating effector function in exhausted memory T cells. Considering the previous observation of differential increases in CD4 and CD8 T cells in the presence of PS mAb, this may contribute to the varying levels of protection against M.tb growth seen in the two LTBI PBMCs. Furthermore, this method can be utilized to determine the optimal therapeutic dose of PS mAb for each individual exposed to M.tb.

### Polyclonal Plasma Antibodies Identified With PS Targeting Antibodies can Enhance the Effector Function of Antigen-Stimulated Memory T Cells

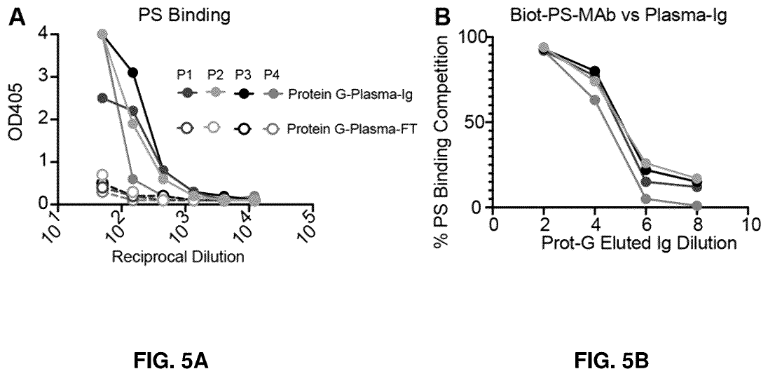

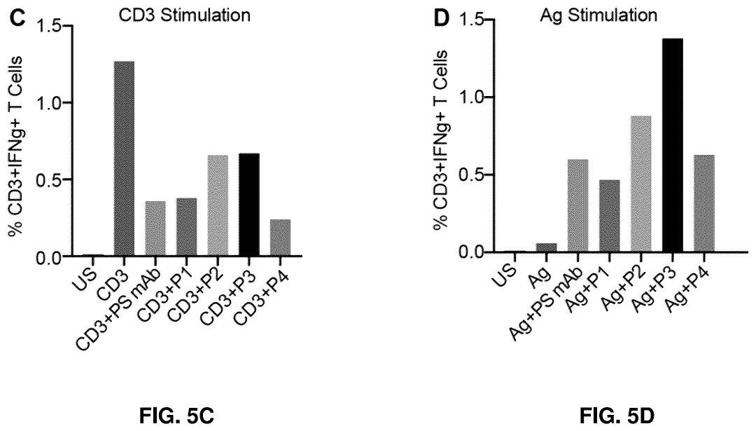

The present discovery of a novel function of PS targeting antibodies in restoring effector function in exhausted or antigen non-responsive memory T cells has shifted the understanding of innate immune responses. It was investigated whether these diverse PS binder antibodies, which can be induced in pathological conditions, have varying effects on the memory T cells when bound to PS either directly or indirectly. To test this, four plasma samples known to contain PS-binding antibodies were used. The total antibodies were purified using a protein G column and divided into two fractions: purified-Ig and flow-through (minus Ig). An ELISA assay was then conducted against PS and confirmed no loss of plasma Ab binding to PS by the eluted plasma Ig fraction which exhibited varying levels of binding to PS, while no binding was observed in the flow-through fraction (FIG. 5A). The PS-binding ability of the eluted plasma Ig was then further confirmed by demonstrating their ability to compete with biotinylated anti-PS mAb for binding to PS-coated ELISA wells (FIG. 5B). Interestingly, while the strength of binding to PS varied among the plasma Ig samples, all of them showed comparable inhibition of anti-PS mAb binding (FIG. 5A-B). Next, whether these plasma Ig samples had similar or different effects on CD3-and antigen-stimulated T cells was investigated. To test this, 100 μL of a 10-fold diluted purified plasma Ig sample from each of the four individuals (P1 to P4) was added to a total of 200 μL activation volume of CD3-and vaccine-antigen-activated T cells isolated from a yearly vaccinated individual. Interestingly, it was observed that all four plasma Ig samples reduced the number of CD3-activated T cells to varying degrees, while simultaneously increasing the number of antigen-activated memory T cells, as detected by flow cytometry for CD3+ cells producing intracellular IFNγ (FIG. 5C-D).

### Treatment With Anti-PS mAb 11.31 Differently Modulated Checkpoint Expression in Unactivated, Antigen-Activated, and CD3/28-Stimulated T Cells Compared to the Isotype Control

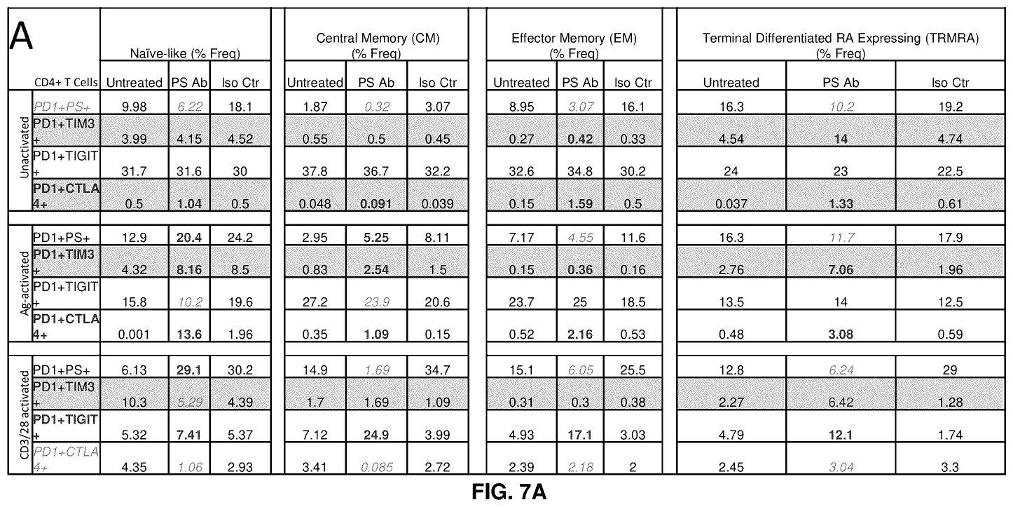

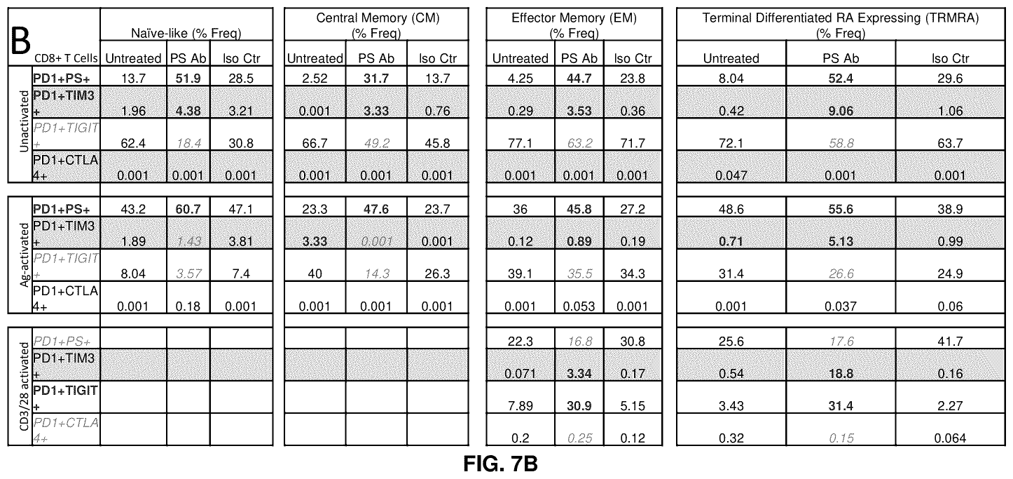

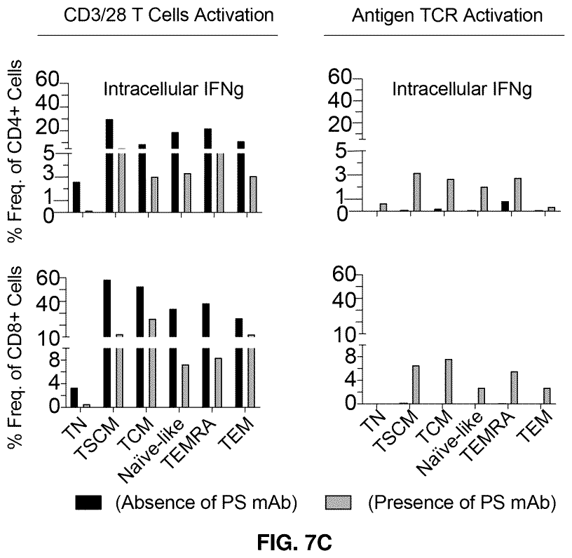

CD4 and CD8 T cells were categorized into four subpopulations based on the differential expression of CD45RA, CCR7, and CD45RO: naïve-like, central memory, effector memory, and terminally differentiated effector memory T cells (see tables at FIGS. 7A and 7B). The frequency of PD1 double-positive cells as a marker for exhausted T cells were evaluated. The findings demonstrated that treatment with anti-PS mAb 11.31 differently modulated checkpoint expression in unactivated, antigen-activated, and CD3/28-stimulated T cells compared to the isotype control. Despite these checkpoint modulations, no intracellular IFNγ was detected in T cells without antigen or CD3/28 stimulation. However, PS antibody significantly enhanced the frequency of antigen-activated T cells with intracellular IFNγ, while reducing CD3/28-activated T cell effector functions (FIG. 7C).

Notably, PS antibody treatment increased PD1+TIM3+ and PD1+CTLA40+ frequencies and decreased PD1+TIGIT+ frequencies among antigen-activated CD4+ T cells. Conversely, PD1+CTLA4 decreased and PD1+TIGIT increased under PS antibody treatment among CD3/28-activated CD4+ T cells (FIG. 7A). In CD8+ T cells, PS antibody treatment of antigen-activated T cells led to increased PD1+PS+ and decreased PD1+TIGIT+ cells, while CD3-activated T cells showed a pronounced increase in PD1+TIGIT+ and a decrease in PD1+PS+ cells (FIG. 7B). The results indicate that the opposing effects in PD1 double-positive populations between antigen and CD3/28 cells can play a crucial role in the PS antibody-mediated modulation of functional T cells upon antigen and CD3/28 stimulation.

TIGIT can be an inhibitory receptor that competes with CD226 to bind CD155 and CD112 on antigen-presenting cells. These findings suggest that anti-PS antibody modulation of TIGIT expression can influence the regulation of both CD3 and antigen-activated T cells, highlighting its potential role in immune modulation in chronic infection and autoimmune diseases.

Finally, FIGS. 6A-6J display graphs of HIV-1 infected (left panel) and TB infected (right panel) plasma Ab binding to PS (FIGS. 6A-6B), CL (FIGS. 6C-6D) and β2GP (FIGS. 6E-6F) as detected by ELISA. A positive correlation was shown between plasma-PS and CL Abs (FIGS. 6G-6H) and PS and CL plasma Ab titer in PTB and LTB patients (FIGS. 6I-6J).

In accordance with one or more embodiments, exemplary methods related to PS-binding biomolecules as described herein are set out in the following items.

Item 1. A method of restoring effector function in antigen non-responsive or exhausted memory T cells, comprising:
delivering a therapeutically effective amount of phosphatidylserine (PS)-binding biomolecules to one or more peripheral blood mononuclear cells (PBMCs) or whole blood of a subject in the presence of antigens specific to one or more diseases or neoantigens specific to cancer cells, wherein the PBMCs or whole blood comprise memory T cells that are exhausted or non-responsive to the antigens specific to the disease or the neoantigens specific to cancer cells;
wherein delivery of the PS-binding biomolecules to the PBMCs or the whole blood restores effector function in the antigen-nonresponsive or neoantigen-nonresponsive memory T cells.

Item 2. The method of item 1, further comprising:
quantifying the increase in effector function of the memory T cells.

Item 3. The method of item 1 or 2, wherein the subject is co-infected with HIV and Mycobacterium tuberculosis (M.tb), and wherein the antigens are HIV—and M.tb-specific antigens, or wherein the subject is exposed to Mycobacterium tuberculosis (M.tb), and wherein the antigens are M.tb-specific antigens.

Item 4. The method of item 1 or 2, wherein the memory T cells are CD4+ memory T cells or CD8+ memory T cells.

Item 5. The method of item 1 or 2, wherein the subject has previously taken a vaccine for the disease.

Item 6. The method of item 1 or 2, wherein the disease is caused by SARS-CoV2 or Mycobacterium tuberculosis.

Item 7. The method of item 1 or 2, wherein the PS-binding biomolecules are antibodies.

Item 8. The method of item 7, wherein the antibodies are monoclonal antibodies.

Item 9. The method of item 7, wherein the antibodies are polyclonal antibodies.

Item 10. The method of item 1, wherein the PS-binding biomolecules are annexins.

Item 11. The method of item 1, wherein the PS-binding biomolecules are synthetic small molecules.

Item 12. A method of diagnosing a latent Mycobacterium tuberculosis (M.tb) infection in a subject, comprising:
obtaining a blood sample of a subject, wherein the blood sample comprises memory T cells;
assaying the blood sample of the subject with PS-binding biomolecules in the presence of M.tb-specific antigens;
measuring a level of IFNγ in the blood sample via the assay; and
determining, based on the level of IFNγ in the assay, whether the subject has a latent M.tb infection.

Item 13. The method of item 12, wherein if the level of IFNγ in the blood sample is IFNγ ≥0.35 IU/ml and ≥25% of Nil, the subject has a latent M.tb infection.

Item 14. The method of item 12 or 13, wherein the assay is a QuantiFERON-TB Gold Plus assay, an Interferon Gamma Release Assay (IGRA), or an ELISPOT assay.

Item 15. The method of any one of items 12-14, wherein the memory T cells are CD4+ memory T cells or CD8+ memory T cells.

Item 16. The method of any one of items 12-14, wherein the PS-binding biomolecules are antibodies.

Item 17. The method of item 16, wherein the antibodies are monoclonal antibodies.

Item 18. The method of item 16, wherein the antibodies are polyclonal antibodies.

Item 19. The method of any one of items 12-14, wherein the PS-binding biomolecules are annexins.

Item 20. The method of any one of items 12-14, wherein the PS-binding biomolecules are synthetic small molecules.

Item 21. A method of enhancing CD4/CD8 memory T cell response in a subject with a Mycobacterium tuberculosis (M.tb) infection, comprising:
administering a therapeutically effective amount of PS-binding molecules to the subject with the M.tb infection in the presence of M.tb-specific antigens.

Item 22. The method of item 21, wherein the memory T cells are CD4+ memory T cells or CD8+ memory T cells.

Item 23. The method of item 21, wherein the PS-binding biomolecules are antibodies.

Item 24. The method of item 23, wherein the antibodies are monoclonal antibodies.

Item 25. The method of item 23, wherein the antibodies are polyclonal antibodies.

Item 26. The method of item 21, wherein the PS-binding biomolecules are annexins.

Item 27. The method of item 21, wherein the PS-binding biomolecules are synthetic small molecules.

Item 28. The method of item 21, wherein the subject is co-infected with M.tb and HIV-1 and wherein the PS-binding molecules are administered in the presence of M.tb-specific and HIV-1 specific antigens.

Item 29. A method of treating one or more active or latent infections in a subject, comprising:
administering a therapeutically effective amount of phosphatidylserine (PS)-binding biomolecules to the subject in the presence of antigens specific to one or more diseases that cause the one or more latent or active infections;
wherein administration of the PS-binding biomolecules restores effector function in memory T cells of the subject that are exhausted or non-responsive to the disease-specific antigens.

Item 30. The method of item 29, wherein the active or latent infection is caused by HIV or Mycobacterium tuberculosis (M.tb).

Item 31. The method of item 29, wherein the subject is co-infected by HIV and M.tb.

Item 32. The method of item 29, wherein the memory T cells are CD4+ memory T cells or CD8+ memory T cells.

Item 33. The method of item 29, wherein the PS-binding biomolecules are antibodies.

Item 34. The method of item 33, wherein the antibodies are monoclonal antibodies.

Item 35. The method of item 33, wherein the antibodies are polyclonal antibodies.

Item 36. The method of item 29, wherein the PS-binding biomolecules are annexins.

Item 37. The method of item 29, wherein the PS-binding biomolecules are synthetic small molecules.

Item 38. The method of item 33, wherein the therapeutically effective amount of the antibodies is approximately 100-200 mg/ml.

Item 39. A method of restoring effector function in antigen non-responsive or exhausted memory T cells of a subject, comprising:
obtaining a blood sample;
delivering a therapeutically effective amount of phosphatidylserine (PS)-binding biomolecules to the blood sample in the presence of antigens specific to one or more diseases or neoantigens specific to cancer cells; and
transfusing the blood sample comprising the phosphatidylserine (PS)-binding biomolecules in the presence of the antigens or neoantigens to the subject,
wherein the transfused blood sample restores effector function in memory T cells of the subject that are exhausted or non-responsive to the antigens specific to one or more diseases or the neoantigens specific to cancer cells.

Item 40. The method of item 39, wherein the antigens are M.tb-specific antigens or HIV-1-specific antigens.

Item 41. The method of item 39, wherein the antigens are SARS-CoV2-specific antigens or neoantigens.

Item 42. The method any one of items 39-41, wherein the PS-binding biomolecules are antibodies.

Item 43. The method of item 42, wherein the antibodies are monoclonal antibodies.

Item 44. The method of item 42, wherein the antibodies are polyclonal antibodies.

Item 45. The method of any one of items 39-41, wherein the PS-binding biomolecules are annexins.

Item 46. The method of any one of items 39-41, wherein the PS-binding biomolecules are synthetic small molecules.

Item 47. A method of treating one or more active infections in a subject, comprising:
administering a therapeutically effective amount of soluble TIGIT alone or in combination with TIM3 to the subject with high titer of plasma PS antibodies to resolve inflammation and T cells exhaustion in the subject.

Item 48. The method of item 47, wherein the subject is co-infected by HIV and M.tb or with autoimmune disease.

Item 49. A method of using PS antibody as adjuvants with vaccine immunogens to induce an effective T cell response.

### REFERENCES

1. van den Eijnde SM, Boshart L, Baehrecke EH, De Zeeuw CI, Reutelingsperger CP, Vermeij-Keers C. Cell surface exposure of phosphatidylserine during apoptosis is phylogenetically conserved. Apoptosis. 1998;3(1):9-16.
2. Schlegel RA, Williamson P. Phosphatidylserine, a death knell. Cell Death Differ. 2001;8(6):551-63.
3. Segawa K, Kurata S, Yanagihashi Y, Brummelkamp TR, Matsuda F, Nagata S. Caspase-mediated cleavage of phospholipid flippase for apoptotic phosphatidylserine exposure. Science. 2014;344(6188):1164-8.
4. Nagata S, Suzuki J, Segawa K, Fujii T. Exposure of phosphatidylserine on the cell surface. Cell Death Differ. 2016;23(6):952-61.
5. Nagata S. Apoptosis and Clearance of Apoptotic Cells. Annu Rev Immunol. 2018;36:489-517.
6. Bettache N, Gaffet P, Allegre N, Maurin L, Toti F, Freyssinet JM, et al. Impaired redistribution of aminophospholipids with distinctive cell shape change during Ca2+-induced activation of platelets from a patient with Scott syndrome. Br J Haematol. 1998;101(1):50-8.
7. Frasch SC, Henson PM, Nagaosa K, Fessler MB, Borregaard N, Bratton DL. Phospholipid flip-flop and phospholipid scramblase 1 (PLSCR1) co-localize to uropod rafts in formylated Met-Leu-Phe-stimulated neutrophils. J Biol Chem. 2004;279(17):17625-33.
8. Dillon SR, Mancini M, Rosen A, Schlissel MS. Annexin V binds to viable B cells and colocalizes with a marker of lipid rafts upon B cell receptor activation. J Immunol. 2000;164(3):1322-32.
9. Sen J, Rosenberg N, Burakoff SJ. Expression and ontogeny of CD2 on murine B cells. J Immunol. 1990;144(8):2925-30.
10. Segawa K, Suzuki J, Nagata S. Constitutive exposure of phosphatidylserine on viable cells. Proc Natl Acad Sci U S A. 2011;108(48):19246-51.
11. Williamson P, Christie A, Kohlin T, Schlegel RA, Comfurius P, Harmsma M, et al. Phospholipid scramblase activation pathways in lymphocytes. Biochemistry. 2001;40(27):8065-72.
12. Hemker HC, van Rijn JL, Rosing J, van Dieijen G, Bevers EM, Zwaal RF. Platelet membrane involvement in blood coagulation. Blood Cells. 1983;9(2):303-17.
13. Kato N, Nakanishi M, Hirashima N. Transbilayer asymmetry of phospholipids in the plasma membrane regulates exocytotic release in mast cells. Biochemistry. 2002;41(25):8068-74.
14. Manodori AB, Barabino GA, Lubin BH, Kuypers FA. Adherence of phosphatidylserine-exposing erythrocytes to endothelial matrix thrombospondin. Blood. 2000;95(4):1293-300.
15. Fischer K, Voelkl S, Berger J, Andreesen R, Pomorski T, Mackensen A. Antigen recognition induces phosphatidylserine exposure on the cell surface of human CD8+ T cells. Blood. 2006;108(13):4094-101.
16. Omi J, Kato T, Yoshihama Y, Sawada K, Kono N, Aoki J. Phosphatidylserine synthesis controls oncogenic B cell receptor signaling in B cell lymphoma. J Cell Biol. 2024;223(2).
17. Gavin MA, Gragerov A, Espling E, Rohde A, Sexton T, Doulami C, et al. Phosphatidylserine Suppresses T Cells Through GPR174, and Co-Inhibition of Adenosine Receptors and GPR174 Synergistically Enhances Th1 Cytokine Production. Annals of Oncology. 2019;30:xi40.
18. Bhatta M, Shenoy GN, Loyall JL, Gray BD, Bapardekar M, Conway A, et al. Novel phosphatidylserine-binding molecule enhances antitumor T-cell responses by targeting immunosuppressive exosomes in human tumor microenvironments. J Immunother Cancer. 2021;9(10).
19. Maclean C, Flegg PJ, Kilpatrick DC. Anti-cardiolipin antibodies and HIV infection. Clin Exp Immunol. 1990;81(2):263-6.
20. Rugman FP, Pinn G, Palmer MF, Waite M, Hay CR. Anticardiolipin antibodies in leptospirosis. J Clin Pathol. 1991;44(6):517-9.
21. Goodridge A, Cueva C, Lahiff M, Muzanye G, Johnson JL, Nahid P, et al. Anti-phospholipid antibody levels as biomarker for monitoring tuberculosis treatment response. Tuberculosis (Edinb). 2012;92(3):243-7.
22. Shen CY, Hsieh SC, Yu CL, Wang JY, Lee LN, Yu CJ. Autoantibody prevalence in active tuberculosis: reactive or pathognomonic? BMJ Open. 2013;3(7).
23. Sarici SU, Gursel O, Kurekci E, Kesik V, Atay A, Okutan V, et al. Anticardiolipin Antibodies in Children with Helicobacter pylori Infection. Helicobacter. 2015;20(6):418-21.
24. Abdel-Wahab N, Lopez-Olivo MA, Pinto-Patarroyo GP, Suarez-Almazor ME. Systematic review of case reports of antiphospholipid syndrome following infection. Lupus. 2016;25(14):1520-31.
25. Khogeer H, Alfattani A, Al Kaff M, Al Shehri T, Khojah O, Owaidah T. Antiphosphatidylserine antibodies as diagnostic indicators of antiphospholipid syndrome. Lupus. 2015;24(2):186-90.
26. Fernandez-Arias C, Rivera-Correa J, Gallego-Delgado J, Rudlaff R, Fernandez C, Roussel C, et al. Anti-Self Phosphatidylserine Antibodies Recognize Uninfected Erythrocytes Promoting Malarial Anemia. Cell Host Microbe. 2016;19(2):194-203.
27. Cheng MP, Butler-Laporte G, Parkes LO, Bold TD, Fritzler MJ, Behr MA. Prevalence of Auto-antibodies in Pulmonary Tuberculosis. Open Forum Infect Dis. 2019;6(4):ofz114.
28. Stelzer M, Henes J, Saur S. The Role of Antiphospholipid Antibodies in COVID-19. Curr Rheumatol Rep. 2021;23(9):72.
29. Melayah S, Kallala O, Ben Ahmed M, Fodha I, Yacoub Jemni S, Ghedira I, et al. IgA anti-beta-2 glycoprotein I antibodies in chronic hepatitis C. Arab J Gastroenterol. 2022;23(1):26-31.
30. Mankai A, Melayah S, Bousetta S, Ghozzi M, Yacoub-Jemni S, Ghedira I. Antiphospholipid antibodies in autoimmune thyroid diseases. J Clin Lab Anal. 2022;36(12):e24788.
31. Yin Y, Huang X, Lynn KD, Thorpe PE. Phosphatidylserine-targeting antibody induces M1 macrophage polarization and promotes myeloid-derived suppressor cell differentiation. Cancer Immunol Res. 2013;1(4):256-68.
32. Moody MA, Liao HX, Alam SM, Scearce RM, Plonk MK, Kozink DM, et al. Anti-phospholipid human monoclonal antibodies inhibit CCR5-tropic HIV-1 and induce beta-chemokines. J Exp Med. 2010;207(4):763-76.
33. Soares MM, King SW, Thorpe PE. Targeting inside-out phosphatidylserine as a therapeutic strategy for viral diseases. Nat Med. 2008;14(12):1357-62.
34. Dowall SD, Graham VA, Corbin-Lickfett K, Empig C, Schlunegger K, Bruce CB, et al. Effective binding of a phosphatidylserine-targeting antibody to Ebola virus infected cells and purified virions. J Immunol Res. 2015;2015:347903.
35. Wang SS, Yan YS, Ho K. US FDA-approved therapeutic antibodies with high-concentration formulation: summaries and perspectives. Antib Ther. 2021;4(4):262-72.
36. Calianese D, Kreiss T, Kasikara C, Davra V, Lahey KC, Gadiyar V, et al. Phosphatidylserine-Targeting Monoclonal Antibodies Exhibit Distinct Biochemical and Cellular Effects on Anti-CD3/CD28-Stimulated T Cell IFN-gamma and TNF-alpha Production. J Immunol. 2021;207(2):436-48.

All publications, patents, and patent documents are incorporated by reference herein, as though individually incorporated by reference. This statement of incorporation by reference is intended by applicants, pursuant to 37 C.F.R. § 1.57(b)(1), to relate to each and every individual publication, patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. No limitations inconsistent with this disclosure are to be understood therefrom.

The invention has been described with reference to various specific and preferred embodiments and techniques. However, it should be understood that many variations and modifications may be made while remaining within the spirit and scope of the invention.

While specific embodiments have been described above with reference to the disclosed embodiments and examples, such embodiments are only illustrative and do not limit the scope of the invention.

What is claimed:

1. A method of restoring effector function in antigen non-responsive or exhausted memory T cells, comprising:
delivering a therapeutically effective amount of phosphatidylserine (PS)-binding biomolecules to one or more peripheral blood mononuclear cells (PBMCs) or whole blood of a subject in the presence of antigens specific to one or more diseases or neoantigens specific to cancer cells, wherein the PBMCs or whole blood comprise memory T cells that are exhausted or non-responsive to the antigens specific to the disease or the neoantigens specific to cancer cells;
wherein delivery of the PS-binding biomolecules to the PBMCs or the whole blood restores effector function in the antigen-nonresponsive or neoantigen-nonresponsive memory T cells.

2. The method of claim 1, further comprising:
quantifying the increase in effector function of the memory T cells.

3. The method of claim 1, wherein the subject is exposed to Mycobacterium tuberculosis (M.tb), and wherein the antigens are M.tb-specific antigens.

4. The method of claim 1, wherein the memory T cells are CD4+ memory T cells or CD8+ memory T cells.

5. The method of claim 1, wherein the subject has previously taken a vaccine for the disease.

6. The method of claim 1, wherein the disease is caused by SARS-CoV2 or Mycobacterium tuberculosis.

7. The method of claim 1, wherein the PS-binding biomolecules are antibodies and wherein the antibodies are monoclonal antibodies or polyclonal antibodies.

8. The method of claim 1, wherein the PS-binding biomolecules are annexins or synthetic small molecules.

9. A method of treating one or more latent infections in a subject, comprising:
administering a therapeutically effective amount of phosphatidylserine (PS)-binding biomolecules to the subject in the presence of antigens specific to one or more diseases that cause the one or more latent infections;
wherein administration of the PS-binding biomolecules restores effector function in memory T cells of the subject that are exhausted or non-responsive to the disease-specific antigens.

10. The method of claim 9, wherein the latent infection is caused by Mycobacterium tuberculosis (M.tb).

11. The method of claim 9, wherein the subject is co-infected by HIV and M.tb.

12. The method of claim 9, wherein the memory T cells are CD4+ memory T cells or CD8+ memory T cells.

13. The method of claim 9, wherein the PS-binding biomolecules are antibodies and wherein the antibodies are monoclonal antibodies or polyclonal antibodies.

14. The method of claim 13, wherein the therapeutically effective amount of the antibodies is approximately 100-200 mg/ml.

15. The method of claim 9, wherein the PS-binding biomolecules are annexins or synthetic small molecules.

16. A method of restoring effector function in antigen non-responsive or exhausted memory T cells of a subject, comprising:
obtaining a blood sample;
delivering a therapeutically effective amount of phosphatidylserine (PS)-binding biomolecules to the blood sample in the presence of antigens specific to one or more diseases or neoantigens specific to cancer cells; and
transfusing the blood sample comprising the phosphatidylserine (PS)-binding biomolecules in the presence of the antigens or neoantigens to the subject,
wherein the transfused blood sample restores effector function in memory T cells of the subject that are exhausted or non-responsive to the antigens specific to one or more diseases or the neoantigens specific to cancer cells.

17. The method of claim 16, wherein the antigens are M.tb-specific antigens.

18. The method of claim 16, wherein the antigens are neoantigens.

19. The method of claim 16, wherein the PS-binding biomolecules are antibodies and wherein the antibodies are monoclonal antibodies or polyclonal antibodies.

20. The method of claim 16, wherein the PS-binding biomolecules are annexins or synthetic small molecules.

21. A method of diagnosing a latent Mycobacterium tuberculosis (M.tb) infection in a subject, comprising:
obtaining a blood sample of a subject, wherein the blood sample comprises memory T cells;
assaying the blood sample of the subject with PS-binding biomolecules in the presence of M.tb-specific antigens;
measuring a level of IFNγ in the blood sample via the assay; and
determining, based on the level of IFNγ in the assay, whether the subject has a latent M.tb infection, wherein if the level of IFNγ in the blood sample is IFNγ≥0.35 IU/ml and ≥25% of Nil, the subject has a latent M.tb infection.
